(19) United States
(12) Patent Application Publication
(10) Pub. No.: US 2025/0240896 A1
(43) Pub. Date: Jul. 24, 2025

(54) PRINTED ELECTRODE ASSEMBLY

(71) Applicant: Coloplast A/S, Humlebaek (DK)

(72) Inventors: Anders Grove Sund, Alleroed (DK); Lars F. Molzen, Alleroed (DK); Carsten Sletten, Espergaerde (DK)

(21) Appl. No.: 18/852,523

(22) PCT Filed: Mar. 29, 2023
(86) PCT No.: PCT/DK2023/050063

§ 371 Date: Sep. 30, 2024

(30) Foreign Application Priority Data
Mar. 29, 2022 (DK) PA202270146

(51) Int. Cl. H05K 3/00; A61B 5/268; A61N 1/05; H05K 1/09; H05K 1/11; H05K 3/12

(52) U.S. Cl. CPC H05K 3/007; A61B 5/268; A61N 1/05; H05K 1/095; H05K 1/118; H05K 3/1283; A61B 2562/125

(57) ABSTRACT

A method of manufacturing an electrode assembly is disclosed, which includes providing a release substrate, forming an electrode assembly on the release substrate; wherein forming an electrode assembly comprises providing a printable first polymer solution; printing the first polymer solution on the first surface of the release substrate according to a first layout; curing the first polymer solution to form a first polymer film with a proximal side comprising a proximal surface and a distal side comprising a distal surface, the proximal surface facing the first surface of the release substrate; providing a conductive ink; printing the conductive ink on the distal surface of the first polymer according to a second layout defining at least one electrode formed from the conductive ink; and curing the conductive ink.

The present disclosure relates to a method of manufacturing a printed electrode assembly and to a printed electrode assembly obtained according to such a method.

### BRIEF DESCRIPTION OF THE DRAWINGS

The accompanying drawings are included to provide a further understanding of embodiments and are incorporated into and a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1A illustrates a cross-sectional view of an electrode assembly obtained according to an embodiment of the invention;

FIG. 1B illustrates a top view of an electrode assembly obtained according to an embodiment of the invention;

FIG. 2 illustrates a perspective view of an electrode assembly obtained according to an embodiment of the invention;

FIG. 3 illustrates a top view of an electrode assembly obtained according to an embodiment of the invention;

FIG. 4 illustrates a top view of an electrode assembly obtained according to an embodiment of the invention;

FIG. 5 illustrates a cross-sectional view of an electrode assembly obtained according to an embodiment of the invention; and

FIG. 6 illustrates a method of manufacturing an electrode assembly according to embodiments of the invention.

### DETAILED DESCRIPTION

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the invention or as a imitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

In the following, whenever referring to proximal side or surface of a layer, an element, a device or part of a device, the referral is to the side or surface facing the release layer, or the side or surface which faced the release layer during manufacturing. Likewise, whenever referring to the distal side or surface of a layer, an element, a device or part of a device, the referral is to the side or surface facing the release layer, or the side or surface which faced the release layer during manufacturing. In other words, the proximal side or surface is the side or surface closest to the release layer during manufacturing and the distal side is the opposite side or surface—the side or surface furthest away from the release layer during manufacturing.

The normal direction is defined as the direction normal/perpendicular to a plane spanned or defined by a substantially planar layer or element.

A radial direction is defined as perpendicular to the normal direction. In particular, the radial direction may be used to describe a direction in the plane of a substantially planar layer or element. In some sentences, the words “inner” and “outer” may be used. These qualifiers should generally be perceived with respect to the radial direction, such that a reference to an “outer” element means that the element is farther away from a centre portion than an element referenced as “inner”. In addition, “innermost” should be interpreted as the portion of a component forming a centre of the component and/or being adjacent to the centre of the component. In analogy, “outermost” should be interpreted as a portion of a component forming an outer edge or outer contour of a component and/or being adjacent to that outer edge or outer contour.

The use of the word “substantially” as a qualifier to certain features or effects in this disclosure is intended to simply mean that any deviations are within tolerances that would normally be expected by the skilled person in the relevant field.

The use of the word “generally” as a qualifier to certain features or effects in this disclosure is intended to simply mean—for a structural feature: that a majority or major portion of such feature exhibits the characteristic in question, and—for a functional feature or an effect: that a majority of outcomes involving the characteristic provides the effect, but that exceptionally outcomes do not provide the effect.

The present disclosure provides a method of manufacturing an electrode assembly, in particular an electrode assembly manufactured by means of printing, and a printed electrode assembly.

In a first aspect of the invention, a method of manufacturing an electrode assembly is disclosed. The method may include the steps of:
providing a release substrate having a first surface; and
forming an electrode assembly on the first surface of the release substrate;
   wherein forming an electrode assembly on the first surface of the release substrate may include:
providing a printable first polymer solution;
printing the first polymer solution on the first surface of the release substrate according to a first layout;
optionally, curing the first polymer solution to form a first polymer film with a proximal side including a proximal surface and a distal side including a distal surface, the proximal surface facing the first surface of the release substrate;
providing a conductive ink;
printing the conductive ink on the distal surface of the first polymer film according to a second layout defining at least one electrode formed from the conductive ink; and
optionally, curing the conductive ink;
   wherein the first layout may define one or more openings in the first polymer film; and
   wherein the at least one electrode at least partly overlaps the one or more openings in the first polymer film; and
   wherein the at least one electrode may extend from a first area, such as of the distal surface, of the first polymer film to a second area, such as of the distal surface, of the first polymer film separate from the first area, and wherein the one or more openings in the first polymer film are defined in the first area.

The steps of providing a release substrate having a first surface and forming an electrode assembly on the first surface of the release substrate may be combined in a step of forming an electrode assembly on a first surface of a release substrate.

Various examples relate to a method of making an electrode assembly including a polymer and an electrode, one or both of which are printed in respective printing process(es). The electrode assembly may be highly customizable, and the manufacturing may be highly optimised (e.g., by being implementable in a single line of manufacturing).

The method may include providing a release substrate having a first surface. The release substrate may be a silicone-coated liner, such as a silicone-coated paper of polymer film. In an embodiment, the release substrate is hydrophobic. In another embodiment, the release substrate may be a liquid, such as water.

The method may include forming an electrode assembly on the first surface of the release substrate. In some examples, sequential steps of printing and, optionally, curing layers of the electrode assembly may be implemented in forming the electrode assembly (e.g., as described in further detail below).

The method may include providing a printable first polymer solution.

The printable first polymer solution may be a liquid, such as a liquid including a polymer dissolved therein, suitable for printing. The first polymer solution may include one or more polymers dissolved in a solvent. In a preferred embodiment, the one or more polymers are dielectric, i.e., electrically insulating. In embodiments, the polymer solution includes polyurethane (PU). In embodiments, the polymer solution includes an acrylate, such as isobornyl acrylate. In embodiments, the polymer solution is a mix of different polymers. In embodiments, the solvent may be acetone, propane-1,2-diol or other commonly used solvent(s) for polymers. The solvent may be selected so as to be suitable for dissolution of the desired polymers and/or for a subsequent curing step, such as to optimise and/or expedite/accelerate curing. In a preferred embodiment, the solvent is highly volatile at room temperature and pressure, such as to quickly evaporate, and thus cure, at least partly.

The method includes printing the first polymer solution on the first surface of the release substrate according to a first layout. For example, the step of providing a printable first polymer solution may include providing the first polymer solution to a suitable printer configured for 2D or 3D printing.

Printing the first polymer solution may include depositing the first polymer solution on the first surface of the release substrate according to a specified first layout. The first layout defines the overall or resulting extent/shape of the polymer film formed once the polymer solution has been cured. Thus, the first layout defines the shape/extent of the polymer film, including any features along edges or the provision of one or more openings of the polymer film. In other words, the first layout represents the desired shape of resulting polymer film. For example, the first layout may be provided defined by a series of operations for the printer, such that, when executed, the printer produces a shape of the polymer solution on the first surface of the release substrate according to the series of operations, which, when cured, results in a polymer film having a corresponding shape. For example, the first layout may be designed in a computer-aided design (CAD) or other drafting software application, such that the series of corresponding operations are generated therefrom and subsequently sent to the printer for processing.

The first layout may be a 2D layout or a 3D layout. For a 3D layout, the thickness (third dimension) may be engineered by printing the first polymer solution multiple times, such as in between intermediate (e.g., partial) curing steps. This may be included in the first layout. In other words, the first layout may specify how many times the first polymer solution should be printed in the same spot to build a thickness. Further, the first layout may specify intermediate breaks of the printing process, such as to allow for a partly curing of the first polymer solution before printing the same spot a second time. Alternatively, the thickness may be engineered by using a larger mesh during printing, which facilitates the deposition of more polymer solution at once. The printing technique may be screen printing.

The method may include curing the first polymer solution to form a first polymer film. The curing may be partial (e.g., not all solvent has evaporated) or complete (all solvent has evaporated). A partial curing may be performed merely by providing a highly volatile solvent, such that it may evaporate quickly after printing.

The first polymer film may have a proximal side including a proximal surface and a distal side including a distal surface, the proximal surface facing (according to definitions herein) the first surface of the release substrate.

Curing the first polymer solution may include exposing the printed first polymer solution to a chemical process, whereby the first polymer solution is cured and/or the associated solvent is evaporated. For example, curing can be initiated by heat, radiation, electron beams, and/or chemical additives, depending on the polymer and/or solvent used.

A first polymer film may be formed by the curing of the first polymer solution. In other words, the first polymer solution may become a first polymer film as a result of the curing process. As used herein, example films include a coherent film or layer-like material. In particular, the film may be a coherent, flexible and elastic/stretchable sheet material.

As mentioned above, without departing from the scope of the invention, it is appreciated that the step of printing the first polymer solution on the first surface of the release substrate according to a first layout may include printing the first polymer solution multiple times, such as to provide a thicker resultant first polymer film. For example, the first polymer solution may be partly cured (e.g., by means of merely the solvent evaporating quickly due to being highly volatile) in between these multiple printing steps. The first layout may change for these intermediate printing steps of the first polymer solution, such as to form regions of the resultant first polymer film having a greater thickness than other regions.

The method may include providing a conductive ink.

In embodiments, the conductive ink includes an electrically conductive material, such as graphite/carbon, silver, or other commonly used materials for printing. In embodiments, the conductive ink includes one or more of metallic (e.g., silver, copper, gold, titanium, aluminium, stainless steel), ceramic (e.g., ITO), polymeric (e.g., PEDOT, PANI, PPy), and carbonaceous (e.g., carbon black, carbon nanotubes, carbon fibres, graphene, graphite) materials.

The conductive ink may include water, acetate or other suitable solvent(s) commonly used in the field. In embodiments, the conductive ink is created by infusing one or more of the above materials into ink. In a preferred embodiment, the conductive ink includes silver, carbon or a mix of silver and carbon.

The method may include printing the conductive ink on the distal surface of the first polymer film according to a second layout defining at least one electrode, thus formed from the conductive ink. Being the result of a printing process of conductive ink, the at least one electrode may also be denoted a conductive trace. In embodiments, the second layout defines a plurality of electrodes, such as two or more electrodes, such as two, three, four, five, six, seven, eight, or more than eight electrodes.

The steps of providing a conductive ink and printing the conductive ink on the distal surface of the first polymer film may be a single step of printing a conductive ink on the distal surface of the first polymer film.

The conductive ink is printed according to a second layout. The second layout defines the overall or resulting shape of the resulting at least one electrode once the conductive ink has been cured. Thus, the second layout defines the shape or path of the at least one electrode, including any features of this at least one electrode. For example, the second layout may specify how the at least one electrode is arranged relative to the first layout (e.g., the shape of the first polymer film).

Preferably, the second layout is provided entirely within an outer periphery of the first layout. The second layout may represent the desired shape of the at least one electrode.

For example, the second layout may be defined by a series of operations for the printer, such that, when executed, the printer produces a shape of the conductive ink on the distal surface of the first polymer film according to the series of operations, which, when cured, results in at least one electrode having a corresponding shape. For example, the second layout may be designed in a computer-aided design (CAD) or other drafting software application, such that the series of corresponding operations are generated therefrom, and subsequently sent to the printer. The second layout may specify how many times the conductive ink should be printed in the same spot to build a thickness. Further, the second layout may specify intermediate breaks of the printing process, such as to allow for a partly curing of the conductive ink before printing the same spot a second time.

In an embodiment, the at least one electrode has a width of maximum 3 mm. In embodiments, the at least one electrode has a width between 0.1 mm and 3 mm, such as between 0.5 mm and 2 mm. In embodiments, the at least one electrode has an aspect ratio (length:width) of at least 5:1, or at least 10:1, or at least 20:1, or at least 25:1, such that the at least one electrode is substantially elongated, such as to provide a conductive path from one area to a second area.

The method may include curing the conductive ink. Curing the conductive ink may include exposing the printed conductive ink to a chemical process, whereby the conductive ink is cured or hardened. For example, curing can be initiated by heat, radiation, electron beams, and/or chemical additives, depending on the conductive ink used, and the process by which it is manufactured.

The first layout may define one or more openings in the first polymer film. Thus, the first layout may define one or more openings in the first polymer film, such that, during printing of the first polymer solution, one or more openings are formed in the resulting first polymer film (e.g., following curing). The first layout may define an outer periphery, wherein the one or more openings are arranged within the outer periphery. The first layout may define an inner periphery, wherein the one or more openings are arranged outside the inner periphery, such as between the outer periphery and the inner periphery.

In embodiments, each of the one or more openings are circular and has a diameter of maximum 5 mm, such 1 mm, 2 mm, 3 mm, 4 mm, or 5 mm. Each of the one or more openings may have a non-zero minimum diameter/size. In embodiments, the one or more openings are elliptic. In embodiments, the one or more openings are rectangular. In embodiments, the one or more openings are circular, elliptic, and/or rectangular. In an embodiment, each of the one or more openings have area less than 18 mm² (corresponding to a circle having a diameter of 4.8 mm). In embodiments, each of the one or more openings have an area less than 10 mm² (corresponding to a circle having a diameter of 3.6 mm).

In an alternative embodiment, the one or more openings are bigger than 3 mm, such as up to 20 mm, such as 4 mm, 5 mm, 6 mm, 7 mm, 8 mm, 9 mm, 10 mm, 12 mm, 14 mm, 15 mm, 16 mm, 18 mm, or 20 mm. In such alternative embodiments, each of the one or more openings may have an area between 10 mm² and 400 mm², such as between 25 mm² and 200 mm², such as between 50 mm² and 150 mm.

In embodiments, the one or more openings includes two or more openings, such as a plurality of openings. Thus, in an embodiment, the first layout defines a plurality of openings in the first polymer film. In embodiments, the plurality of openings includes at least 10 openings, or at least 20 openings, or at least 30 openings. In embodiments, the plurality of openings includes between 2 and 50 openings, such as between 10 and 50 openings.

The one or more openings provides a passage through the first polymer film from its proximal side to its distal side.

The at least one electrode may at least partly overlap the one or more openings in the first polymer film. In other words, the second layout may define the at least one electrode to at least partly overlap with the one or more openings in the first polymer film. Thus, the second layout may specify that the conductive ink at least partly overlaps the one or more openings in the first polymer film. Thereby, a conductive path from the proximal surface to the distal surface is formed, namely via the one or more openings of the first polymer film. In particular, when printing the conductive ink on the first polymer film according to the second layout, which specifies that some of said conductive ink should at least partly overlap the one or more openings, the inventors have found that some of said conductive ink may fill such one or more openings. Thereby, the one or more openings are filled with conductive ink, such that the conductive path from the proximal surface to the distal surface of the first polymer film is formed.

The at least one electrode may extend from a first area of the first polymer film to a second area of the first polymer film separate from the first area, wherein the one or more openings in the first polymer film are defined in the first area. In other words, the second layout may define the at least one electrode to extend from the first area to the second area, wherein the first area includes the one or more openings, and wherein the second area is absent of the one or more openings.

Thus, the at least one electrode is provided on the distal surface of the first polymer film such that it extends from an opening in the film and into an area (the second area) absent of openings. Thereby, the electrode may form a path from an opening of the one or more openings to an area distant the one or more openings. Thereby, the electrode may be used to connect electrically to the proximal surface of the first polymer film in the first area from a position in the second area, separate from the first area. For example, the second area may be provided with one or more terminals and/or an interface for electrically connecting the at least one electrode with an electrical circuit, e.g., an electronic device. The interface may include a coupling part for forming a mechanical connection with the electrical circuit, e.g., the electronic device.

Thereby may be formed a printed electrode assembly which may be highly customisable due to the printing process, and which may define one or more openings through which the electrode may be exposed to the proximal side of the first polymer film, while being supported by the same first polymer film. Further, the electrode assembly may further allow for connecting the at least one electrode with a separate electrical circuit on the distal side of the first polymer film at a position distant from the one or more openings.

Manufacturing an electrode assembly according to the methods described herein may facilitate a single-line manufacturing, potentially connected to the line manufacturing of a target substrate or device incorporating the electrode assembly. Further, the use of printing allows for easily adapting the layout of both the first polymer layer and the one or more electrodes (as defined by the second layout) to the specific target substrate or device, without substantially changing the manufacturing line. For example, when incorporated in a single-line manufacturing process of the device incorporating the electrode assembly, the layout of the electrode assembly, and thus the functionality of the target substrate incorporating the electrode assembly, may be designed to change in accordance with mere instructions from a computer program (e.g., to adapt or otherwise change the first and/or second layout with which the first polymer layer and at least one electrode, respectively, are formed) rather than requiring a rebuild of the manufacturing line.

In an embodiment, the method includes defining an interface on the distal side, such as on the distal surface, of first polymer film, the interface including at least one terminal electrically coupled to the at least one electrode in the second area. Thus, the interface may be arranged in the second area as previously defined. Thereby is facilitated that an electrical circuit or electronic device may be coupled to the at least one electrode in the second area via the interface. In embodiments, the at least one terminal is integral with the at least one electrode. For example, the at least terminal may be defined in the second layout, such that the at least one terminal is printed as part of the respective at least one electrode during the same process. In embodiments, the at least one terminal includes a conductive material different from the conductive material of the at least one electrode. For example, the at least one terminal may include (printed) carbon, where the at least one electrode may include (printed) silver, or vice versa.

In an embodiment, the method further includes the steps of:
providing a printable second polymer solution;
printing the second polymer solution on the distal surface of the first polymer film and/or a distal surface of the conductive ink according to a third layout; and
optionally curing the second polymer solution to form a second polymer film with a proximal surface and a distal surface.

In embodiments, the above steps may include the steps of printing a second polymer solution on the distal surface of the first polymer film and/or a distal surface of the conductive ink according to a third layout, and optionally curing the second polymer solution to form a second polymer film with a proximal surface and a distal surface.

Thus, in further steps of the method, a second polymer film is provided/formed on the distal side of the first polymer film. In particular, the second polymer film is provided/formed by means of printing a second polymer solution according to a third layout. Depending on the third layout, some of the second polymer solution may be printed on top of (i.e., on the distal surface of) the conductive ink (thus, on a distal surface of the at least one electrode), thereby at least partially encapsulating said conductive ink between the first polymer film and the second polymer film, or some of the second polymer solution may be printed on top of (i.e., on the distal surface) of the first polymer film.

Embodiments relating to the provision and composition of the first polymer solution discussed above, and the curing thereof, may similarly apply to the second polymer solution. Thus, for example, the second polymer solution may include the same, or different, polymers and solvents than the first polymer solution. In a preferred embodiment, the second polymer film is dielectric/electrically insulating like the first polymer film. In an embodiment, the first polymer solution and the second polymer solution are identical and may thus include the same polymer solution.

The second polymer solution is printed according to a third layout. The definitions of a first layout as discussed previously may apply to the third layout. In an embodiment, an outer periphery of the third layout corresponds to an outer periphery of the first layout. In embodiments where the first layout defines an inner periphery, an inner periphery of the third layout may likewise correspond to such inner periphery of the first layout. Thereby, the second polymer film generally corresponds in shape to the first polymer film. Preferably, the first layout and the third layout are aligned.

In an embodiment, the first layout and the third layout define an encapsulated part of the second layout. In other words, in an embodiment, the first layout and the third layout define an encapsulated part of the at least one electrode (as defined by the second layout). In further other words, the first polymer film and the second polymer film may encapsulate a part of the at least one electrode. The at least one electrode may be exposed in other parts of the first and/or third layout, such that only one or more parts of the at least one electrode are encapsulated.

In an embodiment, the third layout defines one or more openings in the second polymer film, and wherein said one or more openings at least partly overlap the at least one electrode. The one or more openings of the second polymer film may be similar in definition, shape, and/or size as the one or more openings of the first polymer film discussed previously.

By the one or more openings at least partly overlapping the at least one electrode is meant that the one or more openings in the second polymer film expose at least parts of the at least one electrode.

The third layout may define one or more openings in the second polymer film. In other words, the third layout may define one or more openings in the second polymer film, such that, during printing of the second polymer solution, one or more openings are formed in the resulting second polymer film (e.g., following curing). The third layout may define an outer periphery, wherein the one or more openings are arranged within the outer periphery. The third layout may define an inner periphery, wherein the one or more openings are arranged outside the inner periphery, such as between the outer periphery and the inner periphery.

In embodiments, the one or more openings of the second polymer film includes two or more openings/a plurality of openings. Thus, in an embodiment, the third layout defines a plurality of openings in the second polymer film. In embodiments, the plurality of openings includes at least 2 openings, such as two, three four, five, six, seven, either, or more than eight openings. In embodiments, the number of openings of the second polymer correspond to the number of electrodes defined according to the second layout. Thus, the one or more openings in the second polymer film may be configured to define terminals/access points for providing electrical contact with the at least one electrode.

The one or more openings of the second polymer film provide a passage through the second polymer film from its proximal side to its distal side. In particular, the one or more openings provide access to the at least one electrode from a distal side of the second polymer film.

Thereby, due to the arrangement of one or more openings in the first polymer film and in the second polymer film relative to the at least one electrode, a conductive path is provided between the proximal side of the first polymer film and the distal side of the second polymer film. In other words, the relative arrangement of openings in the first and second polymer film and the at least one electrode provides for assessing (e.g., measuring a parameter electrically) the proximal side of the first polymer film from the distal side of the second polymer film via the electrode(s). For example, the proximal side of the first polymer film may be assessed electrically by means of a voltage applied to two or more electrodes from the distal side of the second polymer film, said two or more electrodes being exposed by correspondingly two or more openings in the second polymer film overlapping the two or more electrodes.

In an embodiment, the first polymer film and the second polymer film define an assembly plane and wherein the one or more openings in the second polymer film are offset, in the assembly plane, from the one or more openings in the first polymer film.

In embodiments, the first polymer film and the second polymer film are substantially planar and may be said to span or define a respective first plane and second plane. When the second polymer film is provided on the distal surface of the first polymer film, an assembly plane may be defined, the assembly plane thus including the parallelly arranged first plane and second plane.

In embodiments, the one or more openings in the second polymer film are offset the one or more openings in the first polymer film in the assembly plane. Consequently, the one or more openings in the second polymer film are not aligned/not arranged immediately distal the one or more openings in the first polymer film. Thereby, it is avoided that the proximal side of the first polymer film is exposed to the distal side of the second polymer film via an opening in the first polymer film aligned with an opening in the second polymer film.

In an embodiment, the second polymer film includes a first area aligned with the first area of the first polymer film and a second area aligned with the second area of the first polymer film. Thereby, the previously defined first and second areas of the first polymer film may be translated/mapped to adjacent areas of the second polymer film. Thereby, the defined first and second areas may be considered first and second areas of the electrode assembly as such.

In an embodiment, the one or more openings of the second polymer film are defined in the second area of the second polymer film. As previously disclosed, the one or more openings of the first polymer film are defined in the first area of said first polymer film. Thereby, since the first and second areas of the first polymer film may be translated to like areas of the second polymer film according to the above embodiment, the one or more openings of the second polymer film are offset the one or more openings of the first polymer film. In other words, when considering the entire electrode assembly, the first area of said electrode assembly includes one or more openings defined in the first polymer film, and the second area, separate from the first area, of said electrode assembly includes one or more openings defined in the second polymer film. Thereby, the one or more openings of each polymer film are offset, since the first area and the second area are separate.

Thereby is defined electrical access to the proximal side of the first polymer film in the first area via the at least one electrode and electrical access to said at least one electrode from the distal side of the second polymer film via the one or more openings in the second area. In other words, electrical access to the proximal side of the electrode assembly is provided distant from where an electrical circuit (e.g., an electronic device) may be coupled to the electrode assembly. Thereby, where the electrode assembly is used for sensing a parameter in an environment on the proximal side of the first polymer film, said parameter may be sensed distant from said environment, whereby disturbance to the environment is minimized or avoided. Further, the build of the electrode assembly allows for keeping the proximal side (and environment) of the electrode assembly sterile, while at the same time facilitating measuring parameters of this sterile environment.

In an embodiment, the electrode assembly is stretchable. The conductive ink of the electrode assembly may be stretchable by means of defining a zigzag-path in the direction of intended stretch, but other solutions are envisioned within the scope of the invention. In embodiments, the electrode assembly is stretchable (by the length ΔL from the length at rest LO) by at least 10% relative to the length at rest LO:

LO+ΔL>1.10×LO

Thereby, the electrode assembly is highly customizable to the intended use. For example, thereby, the electrode assembly may be incorporated in a device exposed to stretch.

In embodiments, the first polymer film, the second polymer film and/or the conductive ink are stretchable by at least 20%, 30%, 40%, 50%, 75% or 100% relative to the length at rest LO.

In an embodiment, the method further includes the step of removing the electrode assembly from the release substrate. Thereby, the electrode assembly may be transferred to a target substrate configured to incorporate the electrode assembly.

In an embodiment, the method further includes the step of arranging the electrode assembly on a target substrate.

In embodiments, the target substrate is a substrate exposed to stretch during intended use. In embodiments, the target substrate is an electronic device. In embodiments, the target substrate is a wearable. In embodiments, the target substrate is a medical device. In embodiments, the medical device is for implantation. In embodiments, the medical device is for attachment to the skin of a mammal, such as a wound dressing or an ostomy appliance. In embodiments, the medical device is for insertion, such as intermittent insertion, into the body of a mammal, such as a catheter, such as an intermittent catheter.

In embodiments, the target substrate is arranged on the distal surface of the electrode assembly, corresponding to the distal surface of the second polymer film, prior to removal of the release substrate. In embodiments, the target substrate is arranged on the proximal surface of the electrode assembly, corresponding to the proximal surface of the first polymer film, following removal of the release substrate. It is appreciated that the definitions of the first, second and third layouts may be adapted to the process by which the electrode assembly is arranged in the target substrate. For example, it may be beneficial to certain manufacturing processes that the first polymer solution is printed according to the third layout as otherwise disclosed in relation to the second polymer solution, and that the second polymer solution is printed according to the first layout as otherwise disclosed in relation to the first polymer solution. Further, an interface, as previously disclosed, may be arranged such that an electronic device coupled to the interface is arranged on a distal side of the electrode assembly, and such that the electrode assembly facilitates accessing its proximal surface via the electrode(s). In other words, the interface may be arranged such that an electronic device may be configured to measure a parameter of the target substrate via the electrode assembly, namely from one side to the other.

In a second aspect of the invention, an electrode assembly is disclosed. The electrode assembly includes:
a printed and cured first polymer film with a proximal side including a proximal surface and a distal side including a distal surface, the first polymer film having a first layout;
at least one electrode formed from a conductive ink, the at least one electrode provided on the distal surface of the first polymer film according to a second layout;
wherein first polymer film may include one or more openings; and
wherein the at least one electrode may at least partly overlap the one or more openings in the first polymer layer for providing a conductive path from the proximal surface to the distal surface of the first polymer film; and
wherein the at least one electrode may extend from a first area of the distal surface of the first polymer film to a second area of the distal surface of the first polymer film separate from the first area, and wherein the one or more openings in the first polymer film are arranged in the first area of the distal surface of the first polymer film.

In embodiments, the electrode assembly may be obtained by the method according to the first aspect of the invention.

It is appreciated that embodiments, including definitions, materials, features and benefits, as disclosed in relation to the first aspect of the invention are applicable to an electrode assembly according to the second aspect of the invention. Thus, embodiments of the second aspect of the invention may be obtained by methods of the first aspect of the invention.

### DETAILED DESCRIPTION OF THE DRAWINGS

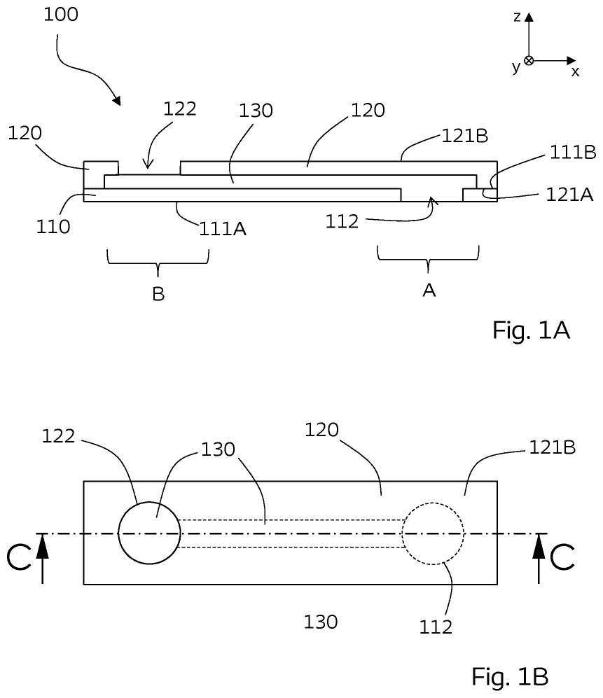

FIG. 1A illustrates a cross-sectional view taken along line C-C of the top view in FIG. 1B of an electrode assembly 100 according to an embodiment of the invention.

The electrode assembly 100 includes a first polymer film 110, a second polymer film 120, and an electrode 130. The first polymer film 110 has a proximal side with a proximal surface 111A and distal side with a distal surface 111B. The second polymer film 120 has a proximal side with a proximal surface 121A and a distal side with a distal surface 121B. The electrode 130 is arranged between the distal surface 111B of the first polymer film 110 and the proximal surface 121A of the second polymer film 120.

The first polymer film 110 is formed after curing of a first polymer solution printed according to a first layout defining the spatial extent/shape of the first polymer film 110. The first layout defines a first primary opening 112 in the first polymer film 110 providing a passageway from the proximal surface 111A to the distal surface 111B of the first polymer film 110.

The second polymer film 120 is formed after curing of a second polymer solution printed according to a third layout defining the spatial extent/shape of the second polymer film 120. The third layout defines a first secondary opening 122 providing a passageway from the proximal surface 121A to the distal surface 121B of the second polymer film 120.

The first primary opening 112 and the first secondary opening 122 are offset each other in the assembly plane as spanned by the (x,y) plane of the indicated coordinate system (x,y,z).

The electrode 130 is formed after curing a conductive ink printed according to a second layout defining the spatial extent/shape of the electrode 130. The second layout defines a conductive path, i.e., the electrode 130, between the first primary opening 112 and the first secondary opening 122. Thereby, the electrode 130 facilitates assessing the proximal surface 111A of the first polymer film 110 via the first secondary opening 122 in the distal surface 121B of the second polymer film 120.

The electrode 130 extends from a first area A in the appliance plane (x,y) to a second area B in the appliance plane (x,y) of the first polymer film 110. The first primary opening 112 is arranged in the first area A. The first secondary opening 122 in the second polymer film 120 is arranged in the second area B.

FIG. 1B illustrates a top view of the distal surface 121B of the second polymer film 120 of the electrode assembly 100 of FIG. 1. The first secondary opening 122 is visible. The electrode 130 is exposed in the opening 122.

In dashed lines, to illustrate otherwise hidden features, are illustrated the first primary opening 112 and the electrode 130 as extending from the first secondary opening 122 to the first primary opening 112. The extent of the electrode 130 and the first primary opening 112 would otherwise not be visible from the distal surface 121B of the second polymer film 120.

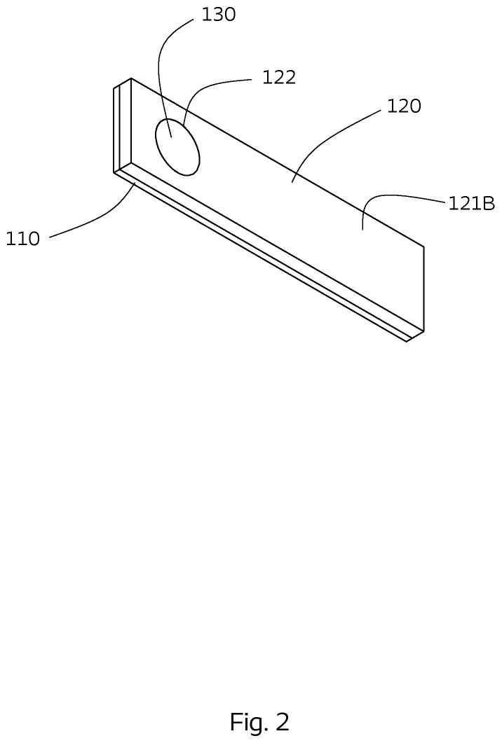

FIG. 2 illustrates a perspective view of the electrode assembly of FIG. 1. In particular, FIG. 2 highlights the distal side and surface 121B of the second polymer film 120. In FIG. 2A, the first secondary opening 122 of the second polymer film 120 is visible, with the electrode 130 exposed in the opening 122. The first polymer film 110 is arranged proximal to the second polymer film 120.

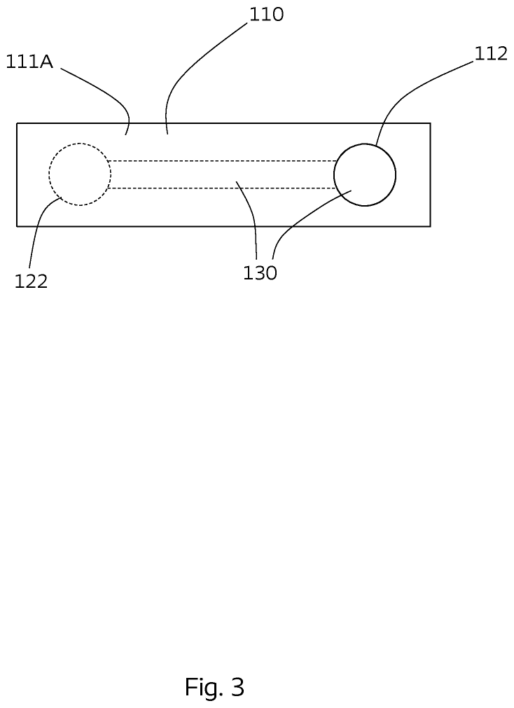

FIG. 3 illustrates a top view of the proximal surface 111A of the first polymer film 110 of the electrode assembly 100 of FIG. 1. The first primary opening 112 is visible. The electrode 130 is exposed in the opening 112.

In dashed lines, to illustrate otherwise hidden features, are illustrated the first secondary opening 122 and the electrode 130 as extending from the first primary opening 112 to the first secondary opening 122. The extent of the electrode 130 and the first secondary opening 122 would otherwise not be visible from the proximal surface 111A of the first polymer film 110.

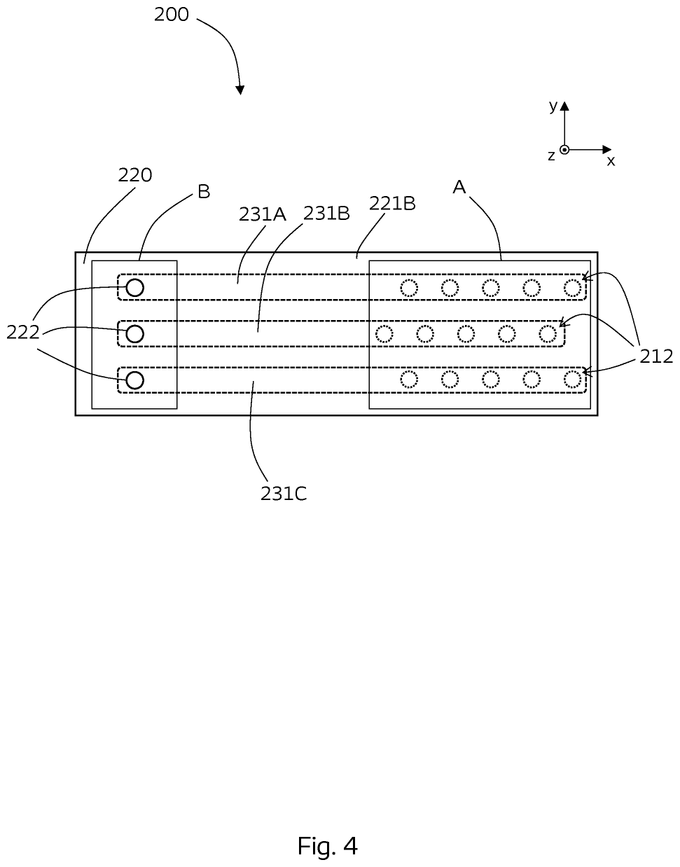

FIG. 4 illustrates a top view of the distal side and distal surface 221B of the second polymer film 220 of an electrode assembly 200 according to an embodiment of the invention. The illustration is drawn with components in dashed lines illustrating features being arranged proximal to the second polymer film 120 in order to highlight the relative arrangement of openings and the electrodes in the normal (z) direction relative to the assembly plane (x,y).

A plurality of first primary openings 212 (dotted circles to highlight arrangement despite being hidden in the present view) are arranged in a first area A of the assembly plane (x,y). The plurality of first primary openings 212 are openings of the first polymer film (which not explicitly illustrated as the figure illustrates the distal side). Thus, the plurality of first primary openings 212 are exposed to the proximal side of the electrode assembly 200.

A plurality of first secondary openings 222 are arranged in a second area B of the assembly plane (x,y). The second area B is separate from the first area A. Thereby, the plurality of first secondary openings 222 are displaced relative to the first primary openings 212 in the assembly plane (x,y). The plurality of first secondary openings 222 are exposed to the distal side of the electrode assembly 200.

A plurality of electrodes including a first electrode 231A, a second electrode 231B, and a third electrode 231C is illustrated (dashed rectangles to highlight arrangement despite being hidden in the present view). Each of the plurality of electrodes 231A, 231B, 231C extends from the first area A to the second area B in the appliance plane (x,y). Each of the plurality of electrodes at least partly overlaps an opening (here, five openings each) of the plurality of first primary openings 212. Further, each of the plurality of electrodes overlaps an opening of the plurality of first secondary openings 222. Thus, each electrode 231A, 231B, 231C extends from an opening of the plurality of first primary openings 212 in the first area A to an opening of the plurality of first secondary openings 222 in the second area B. Thereby, each electrode 231A, 231B, 231C is partly exposed to the proximal side of the first polymer film via first primary openings 212 and to the distal side of the second polymer film 220 via first secondary openings 222. Thereby, the electrodes may (e.g., by means of an electronic device coupled to the electrode) obtain an electrical parameter from the first area A on the proximal side of the electrode assembly 200 when said proximal side of the electrode assembly is exposed to surroundings of interest for sensing, and the electrical parameter may be obtained via the plurality of first secondary openings 222 on the distal side of the electrode assembly 200 from the second area B.

The electrode assembly 200 may be manufactured according to methods described herein, in particular printing methods, whereby the electrode assembly may be manufactured in a single-line process connected to the line manufacturing of the target substrate or device incorporating the electrode assembly 200. Further, the use of printing allows for easily adapting the layout of both the first polymer layer, the electrodes and the second polymer layer to the specific target substrate or device, without substantially changing the manufacturing line. For example, when incorporated in a single-line manufacturing process of the device incorporating the electrode assembly, the layout of the electrode assembly and the functionality of the target substrate, may be designed to change in accordance with mere instructions from a computer program (e.g., including the series of operations for one or more printers) rather than requiring a rebuild of the manufacturing line.

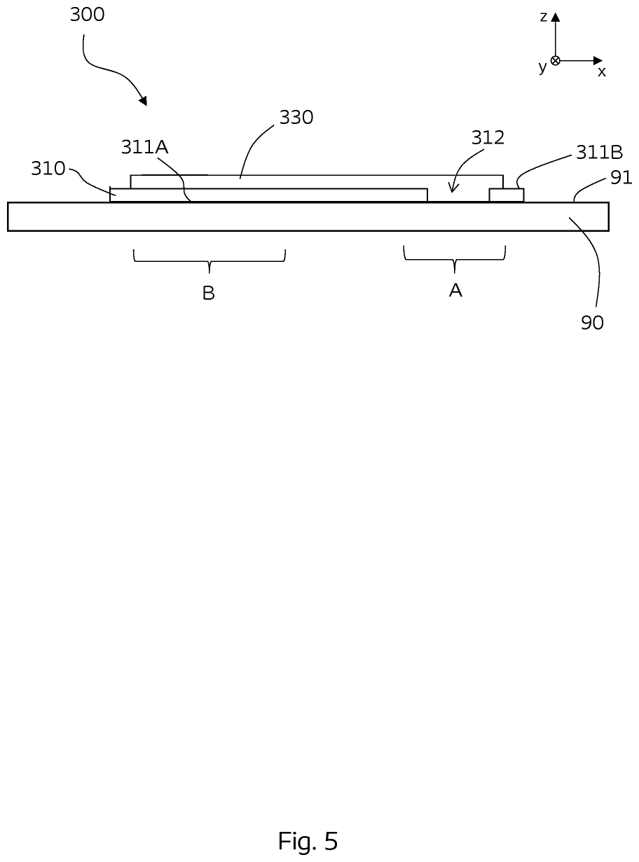

FIG. 5 illustrates a cross-sectional view of an electrode assembly 300 according to an embodiment of the invention. The electrode assembly 300 includes a first polymer film 310 arranged on, such as printed on, a first surface 91 of a release substrate 90. The first polymer film 310 includes a proximal surface 311A facing the first surface 91 and a distal surface 311B.

The first polymer film 310 is formed after curing of a first polymer solution printed on the first surface 91 according to a first layout defining the spatial extent/shape of the first polymer film, including defining the opening 312.

A conductive ink is printed on the distal surface 311B according to a second layout defining the spatial extent/shape of the electrode 330. The electrode 330 is formed from the curing of the conductive ink.

The second layout defines the electrode 330 to at least partly overlap the opening 312 defined in the first polymer film 310. The second layout defines the electrode 330 to extend from a first area A of the first polymer film 310 to a second area B of the first polymer film 310, the second area B being separate from the first area A. The opening 312 in the first polymer film 310 is defined in the first area A.

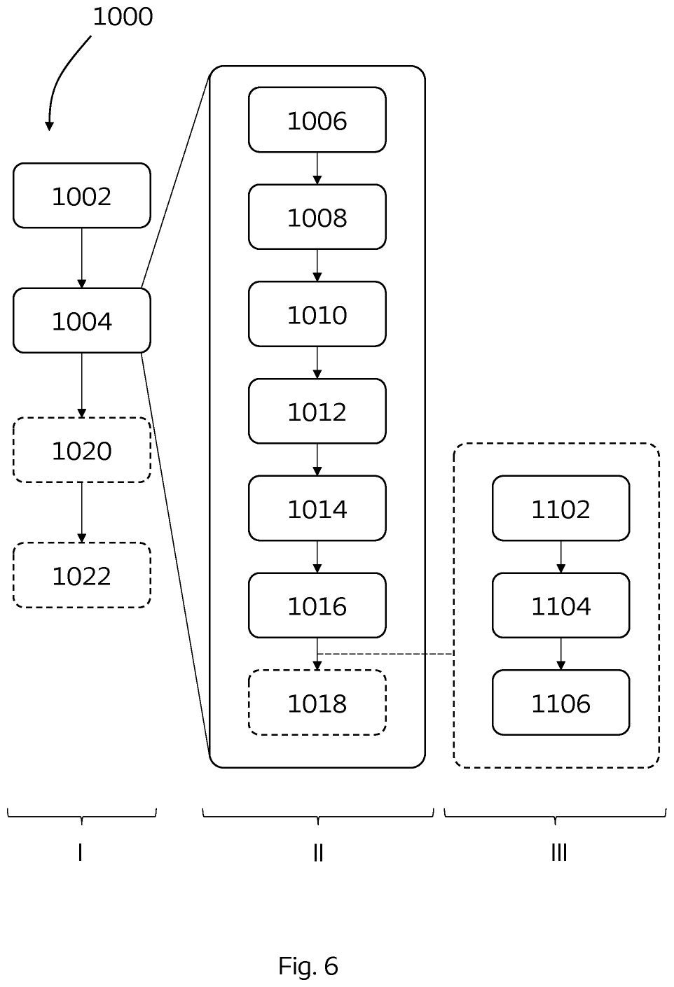

FIG. 6 illustrates a method 1000 of manufacturing an electrode assembly according to embodiments of the invention. The method 1000 includes the steps of
providing 1002 a release substrate having a first surface; and
forming 1004 an electrode assembly on the first surface of the release substrate, as illustrated in the left column I.

The step of forming 1004 an electrode assembly on the first surface of the release substrate includes the steps of:
providing 1006 a printable first polymer solution;
printing 1008 the first polymer solution on the first surface of the release substrate according to a first layout;
curing 1010 the first polymer solution to form a first polymer film with a proximal side including a proximal surface and a distal side including a distal surface, the proximal surface facing the first surface of the release substrate;
providing 1012 a conductive ink;
printing 1014 the conductive ink on the distal surface of the first polymer film according to a second layout defining at least one electrode formed from the conductive ink; and
curing 1016 the conductive ink,
   as illustrated in the centre column II.
   wherein the first layout defines one or more openings in the first polymer film; and wherein the at least one electrode at least partly overlaps the one or more openings in the first polymer film; and wherein the at least one electrode extends from a first area of the first polymer film to a second area of the first polymer film separate from the first area, and wherein the one or more openings in the first polymer film are defined in the first area.

The method 1000 may further include the optional step (dashed) of defining 1018 an interface on the distal surface of the first polymer film, the interface including at least one terminal electrically coupled to the at least one electrode in the second area.

The method 1000 may include further optional steps (dashed). In particular, the further optional steps relate to forming a second polymer film, as illustrated in the right column III. The further optional steps may be provided prior to defining 1018 an interface and include:
providing 1102 a printable second polymer solution;
printing 1104 the second polymer solution on the distal surface of the first polymer film and/or a distal surface of the conductive ink according to a third layout; and
curing 1106 the second polymer solution to form a second polymer film with a proximal surface and a distal surface.

The method 1000 may further include the optional step (dashed) of removing 1020 the electrode assembly from the release substrate and the optional step (dashed) of arranging 1022 the electrode assembly on a target substrate, as illustrated in the left column I.

Dashed boxes of the method 1000 in FIG. 6 are intended to illustrate optional steps of a method.

Whereas the first polymer film and the second polymer film may include different features, such as different layouts, such as different arrangements or numbers of openings, it is appreciated that the first polymer film may be easily adapted to the definitions or layout of the second polymer film and vice versa. Thus, the first polymer film may be printed according to the third layout, and the second polymer film may be printed according to the first layout, without departing from the scope of the invention. Likewise, it is appreciated that the definitions of proximal and distal may be swapped depending on the process incorporating the manufacturing process.

Whereas the individual steps of the method are disclosed as being single steps performed once, it is appreciated that these single steps may be performed multiple times before moving to the next step. For example, it is well-known that a thickness may be introduced by printing the same material on the same spot multiple times. In other words, the disclosure of a step of the method is not a disclosure of said step being performed only once before moving to the next step of the method.

The use of the terms “first”, “second”, “third” and “fourth”, “primary”, “secondary”, “tertiary” etc. does not imply any particular order but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering.

Furthermore, the labelling of a first element does not imply the presence of a second element and vice versa.

It is to be noted that the words “comprising” and “including” do not necessarily exclude the presence of other elements or steps than those listed.

It is to be noted that the words “a” or “an” preceding an element or method step do not exclude the presence of a plurality of such elements or method steps.

Although particular features have been shown and described, it will be understood that they are not intended to limit the claimed invention, and it will be made obvious to those skilled in the art that various changes and modifications may be made without departing from the spirit and scope of the claimed invention. The specification and drawings are, accordingly, to be regarded in an illustrative rather than restrictive sense. The claimed invention is intended to cover all alternatives, modifications, and equivalents.

Exemplary embodiments are set out in the following items:

1. A method of manufacturing an electrode assembly, the method comprising:
printing a first polymer solution on a first surface of a release substrate according to a first layout defining one or more openings in a first polymer film;
curing the first polymer solution, thereby forming the first polymer film comprising:
   a proximal side comprising a proximal surface that faces the first surface of the release substrate; and
   a distal side comprising a distal surface;
printing conductive ink on the distal surface of the first polymer film according to a second layout defining at least one electrode formed from the conductive ink, wherein:
   the at least one electrode at least partly overlaps the one or more openings in the first polymer film;
   the at least one electrode extends from a first area of the first polymer film to a second area of the first polymer film separate from the first area; and
   the one or more openings in the first polymer film are defined in the first area; and
curing the conductive ink, thereby forming an electrode assembly on the distal surface of the first polymer film.

2. The method of item 1, wherein the at least one electrode has a width of maximum 3 mm.

3. The method of item 1, wherein each of the one or more openings have an area less than 18 mm².

4. The method of item 1, further comprising defining an interface on the distal surface of the first polymer film, the interface comprising at least one terminal electrically coupled to the at least one electrode in the second area.

5. The method of item 1, further comprising:
printing, according to a third layout, a second polymer solution on at least one of:
   the distal surface of the first polymer film; and
   a distal surface of the electrode assembly; and
curing the second polymer solution to form a second polymer film with a proximal surface and a distal surface.

6. The method of item 5, wherein an outer periphery of the third layout substantially corresponds to an outer periphery of the first layout.

7. The method of item 5, wherein the third layout and the first layout define an encapsulated part of the second layout.

8. The method of item 5, wherein the third layout defines one or more openings in the second polymer film, and wherein said one or more openings at least partly overlap the at least one electrode.

9. The method of item 8, wherein:
the first polymer film and the second polymer film define an assembly plane; and
the one or more openings in the second polymer film are offset, in the assembly plane, from the one or more openings in the first polymer film.

10. The method of item 9, wherein the second polymer film comprises:
a first area aligned with the first area of the first polymer film; and
a second area aligned with the second area of the first polymer film.

11. The method of item 10, wherein the one or more openings of the second polymer film are defined in the second area of the second polymer film.

12. The method of item 5, wherein the first polymer solution is the second polymer solution.

13. The method of item 1, wherein the electrode assembly is stretchable.

14. The method of item 1, wherein the first surface of the release substrate is hydrophobic.

15. The method of item 1, further comprising removing the electrode assembly from the release substrate.

16. The method of item 15, further comprising arranging the electrode assembly on a target substrate.

17. An electrode assembly comprising:
a first polymer film printed according to a first layout, the first polymer film comprising:
   a proximal side having a proximal surface;
   a distal side having a distal surface, the distal surface comprising a first area and a second area separate from the first area; and
   one or more openings arranged in the first area of the distal surface; and
at least one electrode formed, according to a second layout and from conductive ink, on the distal surface of the first polymer film, wherein:
   the at least one electrode extends from the first area of the first polymer film to the second area of the first polymer film; and
   the at least one electrode at least partly overlaps the one or more openings in the first polymer layer, thereby providing a conductive path from the proximal surface of the first polymer film to the distal surface of the first polymer film.

18. The electrode assembly of item 17, further comprising:
a second polymer film printed according to a third layout on at least one of:
   the distal surface of the first polymer film; and
   a distal surface of the at least one electrode.

19. The electrode assembly of item 18, an outer periphery of the third layout corresponds to an outer periphery of the first layout.

20. The electrode assembly of item 18, wherein the third layout defines one or more openings in the second polymer film that at least partly overlap the at least one electrode.

1. A method of manufacturing an electrode assembly, the method comprising the steps of:
providing a release substrate having a first surface; and
forming an electrode assembly on the first surface of the release substrate;
wherein forming an electrode assembly on the first surface of the release substrate comprises:
providing a printable first polymer solution;
printing the first polymer solution on the first surface of the release substrate according to a first layout;
curing the first polymer solution to form a first polymer film with a proximal side comprising a proximal surface and a distal side comprising a distal surface, the proximal surface facing the first surface of the release substrate;
providing a conductive ink;
printing the conductive ink on the distal surface of the first polymer film according to a second layout defining at least one electrode formed from the conductive ink;
curing the conductive ink;
providing a printable second polymer solution;
printing the second polymer solution on at least one of the distal surface of the first polymer film or a distal surface of the conductive ink according to a third layout; and
curing the second polymer solution to form a second polymer film with a proximal surface and a distal surface;
wherein the first layout defines one or more openings in the first polymer film;
wherein the at least one electrode at least partly overlaps the one or more openings in the first polymer film;
wherein the third layout defines one or more openings in the second polymer film that at least partly overlap the at least one electrode;
wherein the at least one electrode extends from a first area of the first polymer film to a second area of the first polymer film separate from the first area, and wherein the one or more openings in the first polymer film are defined in the first area; and
wherein the first polymer film and the second polymer film define an assembly plane and wherein the one or more openings in the second polymer film are offset, in the assembly plane, from the one or more openings in the first polymer film.

2. The method according to claim 1, wherein the at least one electrode has a width of maximum 3 mm.

3. The method according to claim 1, wherein each of the one or more openings have an area less than 18 mm².

4. The method according to claim 1, wherein the method comprises defining an interface on the distal surface of the first polymer film, the interface comprising at least one terminal electrically coupled to the at least one electrode in the second area.

5. (canceled)

6. The method according to claim 1, wherein an outer periphery of the third layout corresponds to an outer periphery of the first layout.

7. The method according to claim 1, wherein the third layout and the first layout define an encapsulated part of the second layout.

8-9. (canceled)

10. The method according to claim 1, wherein the second polymer film comprises a first area aligned with the first area of the first polymer film and a second area aligned with the second area of the first polymer film.

11. The method according to claim 10, wherein the one or more openings of the second polymer film are defined in the second area of the second polymer film.

12. The method according to claim 1, wherein the first polymer solution and the second polymer solution are identical.

13. The method according to claim 1, wherein the electrode assembly is stretchable.

14. The method according to claim 1, wherein the first surface of the release substrate is hydrophobic.

15. The method according to claim 1, wherein the method further comprises the step of removing the electrode assembly from the release substrate.

16. The method according to claim 15, wherein the method further comprises the step of arranging the electrode assembly on a target substrate.

17. An electrode assembly comprising:
a printed and cured first polymer film with a proximal side comprising a proximal surface and a distal side comprising a distal surface, the first polymer film having a first layout;
at least one electrode formed from a conductive ink, the at least one electrode provided on the distal surface of the first polymer film according to a second layout; and
a printed and cured second polymer film with a proximal surface and a distal surface, and arranged on at least one of the distal surface of the first polymer film or a distal surface of the conductive ink, the second polymer film having a third layout;
wherein the first polymer film comprises one or more openings;
wherein the at least one electrode at least partly overlaps the one or more openings in the first polymer layer for providing a conductive path from the proximal surface to the distal surface of the first polymer film;
wherein the third layout defines one or more openings in the second polymer film that at least partly overlap the at least one electrode;
wherein the at least one electrode extends from a first area of the distal surface of the first polymer film to a second area of the distal surface of the first polymer film separate from the first area, and wherein the one or more openings in the first polymer film are arranged in the first area of the distal surface of the first polymer film; and
wherein the first polymer film and the second polymer film define an assembly plane and
wherein the one or more openings in the second polymer film are offset, in the assembly plane, from the one or more openings in the first polymer film.

18. The electrode assembly according to claim 17, wherein the at least one electrode has a width of maximum 3 mm.

19. The electrode assembly according to claim 17, wherein each of the one or more openings have an area less than 18 mm².

20. The electrode assembly according to claim 17, wherein the second polymer film comprises a first area aligned with the first area of the first polymer film and a second area aligned with the second area of the first polymer film.

21. The electrode assembly according to claim 20, wherein the one or more openings of the second polymer film are defined in the second area of the second polymer film.

22. The electrode assembly according to claim 17, wherein the first polymer solution and the second polymer solution are identical.

23. The electrode assembly according to claim 17, wherein the electrode assembly is stretchable.
